(19) United States
(12) Patent Application Publication
(10) Pub. No.: US 2023/0410381 A1
(43) Pub. Date: Dec. 21, 2023

(54) ZEN FACILITATION SYSTEM AND NON-TRANSITORY COMPUTER-READABLE STORAGE MEDIUM

(71) Applicant: GLOBALTREEHOUSE INC., Minato-ku, Tokyo (JP)

(72) Inventors: Masamichi YAMADA, Minato-ku, Tokyo (JP); Thierry Georges PORTE, Minato-ku, Tokyo (JP); Frederick Katsuro SCHMIDT, Minato-ku, Tokyo (JP)

(73) Assignee: ITOKI CORPORTAION, Osaka (JP)

(21) Appl. No.: 18/247,250

(22) PCT Filed: Sep. 30, 2020
(86) PCT No.: PCT/JP2020/037231

§ 371 Date: Mar. 30, 2023

(51) Int. Cl. G06T 11/00; G06F 3/01; G06F 3/0484; G06F 3/04815

(52) U.S. Cl. CPC G06T 11/00; G06F 3/011; H04L 67/12; G06F 3/04815; G06T 2200/24; G06F 3/0484

(57) ABSTRACT

A Zen facilitation system includes: a first storage part storing a plurality of avatars corresponding to a plurality of members belonging to a group performing Zen; a second storage part storing at least one virtual Zen space; at least one input receiving part receiving an input from each of the members; a display image generation part generating a Zen action image in which some of the plurality of avatars perform Zen in the Zen space in accordance with an input regarding a Zen action through the input receiving part; and a display part displaying a Zen action image generated in the display image generation part.

### TECHNICAL FIELD

The present disclosure relates to a Zen facilitation system and a program.

### BACKGROUND ART

Patent Document 1 discloses a mindfulness support device including a respiration sensor obtaining respiration information indicating a state of a respiration of a user, a respiration information processing part generating assist information of assisting an activity of the user based on the respiration information, and an output part outputting the assist information in a predetermined form in accordance with the assist information. Patent Document 1 exemplifies that Zen is performed as a means of mindfulness.

### PRIOR ART DOCUMENTS

### Patent Document(s)

Patent Document 1: Japanese Patent Application Laid-Open No. 2019-150546

### SUMMARY

### Problem to be Solved by the Invention

Proposed to facilitate a mental activity by Zen is sitting in Zen meditation in a Zen space, such as a Zen meditation hall, which is peace and designed to be suited for performing Zen. However, there is a case where it is difficult to go to the Zen space due to a positional, temporal, or epidemiological limitation, for example.

Accordingly, an object of the present disclosure is to facilitate a mental activity by Zen regardless of a positional, temporal, or epidemiological limitation.

### Means to Solve the Problem

In order to solve the above problem, a Zen facilitation system includes: a first storage part storing a plurality of avatars corresponding to a plurality of members belonging to a group performing Zen; a second storage part storing at least one virtual Zen space; at least one input receiving part receiving an input from each of the members; a display image generation part generating a Zen action image in which some of the plurality of avatars perform Zen in the Zen space in accordance with an input regarding a Zen action through the input receiving part; and a display part displaying a Zen action image generated in the display image generation part.

A program makes a computer execute processing of receiving an input regarding a Zen action from a member belonging to a group performing Zen and processing of displaying, on a display part, a Zen action image in which some of a plurality of avatars corresponding to a plurality of members belonging to the group perform Zen in a virtual Zen space upon receipt of an input regarding a Zen action.

Another program makes a computer execute processing of making a connection communicably with a plurality of terminal apparatuses on a side of a plurality of members belonging to a group performing Zen via communication network; processing of receiving an input regarding a Zen action from the members belonging to the group via the plurality of terminal apparatuses; processing of generating data expressing a Zen action image in which some of a plurality of avatars corresponding to the plurality of members belonging to the group perform Zen in a virtual Zen space upon receipt of an input regarding a Zen action; and processing of transmitting the data expressing the Zen action image to at least one of the plurality of terminal apparatuses.

### Effects of the Invention

According to the Zen facilitation system or the program described above, a mental activity by Zen can be facilitated regardless of a positional, temporal, or epidemiological limitation.

### BRIEF DESCRIPTION OF DRAWINGS

FIG. 1 A block diagram illustrating a whole configuration of a Zen facilitation system according to a first embodiment.

FIG. 2 A block diagram of a server.

FIG. 3 A flow chart illustrating a processing example of the server.

FIG. 4 A block diagram of a terminal apparatus.

FIG. 5 A flow chart illustrating a processing example of the terminal apparatus.

FIG. 6 An explanation diagram illustrating a transition example of an image display of the terminal apparatus.

FIG. 7 A flow chart illustrating processing for generating and displaying a Zen action image.

FIG. 8 An explanation diagram illustrating an example of an avatar and a Zen space.

FIG. 9 A diagram illustrating an example of the Zen action image.

FIG. 10 A diagram illustrating another example of the Zen action image.

FIG. 11 A block diagram of a server according to a second embodiment.

FIG. 12 A block diagram of a terminal apparatus according to the second embodiment.

FIG. 13 A flow chart illustrating processing for generating and displaying a Zen action image according to the second embodiment.

### DESCRIPTION OF EMBODIMENT(S)

### First Embodiment

<Whole Configuration>

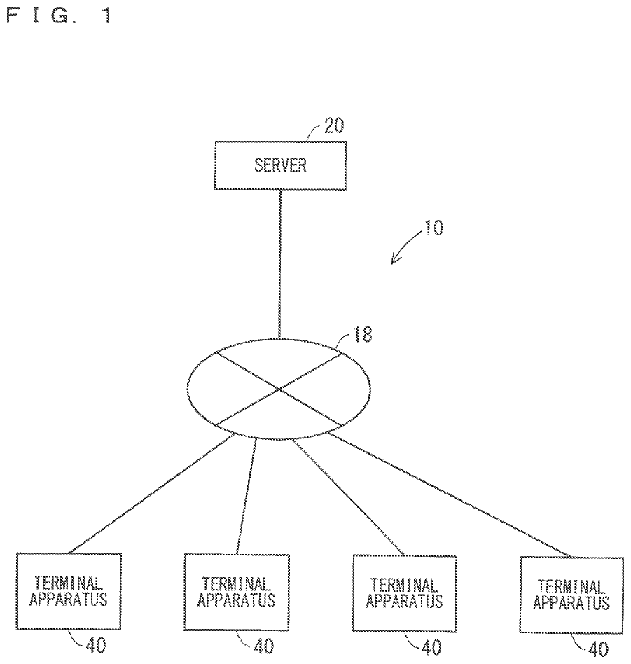

Described hereinafter is a Zen facilitation system and a program according to a first embodiment. FIG. 1 is a block diagram illustrating a whole configuration of the Zen facilitation system. A Zen facilitation system 10 includes a server 20 and a terminal apparatus 40. The terminal apparatus 40 is a terminal apparatus owned by a user. The terminal apparatus 40 may be a smartphone or a tablet computer. The terminal apparatus 40 may be a laptop computer or a desktop personal computer.

The user owning the terminal apparatus 40 is assumed to be a member belonging to a group performing Zen. In the present embodiment, the group is a group in which a plurality of persons intending to perform Zen in a virtual space are registered. Zen is also referred to as Zazen, and is a type of a method of meditation for performing mental concentration while sitting in a cross-legged state. Zen is referred to as Zen Meditation or Zen Buddhism in English. Zen is originally a practice derived from ancient India or Buddhism. Recently, Zen attracts attention as a method having potency of increasing concentration power and attentiveness, reducing a barrier against the other person and causing a sense of unity with the other person, and increasing inspiration and innovation beyond a limit of religion.

Zen may be performed in any location, however, a surrounding environment such as a surrounding landscape, brightness, a degree of peace may have influence on the effects of Zen. Thus, Zen is performed in a Zen space, such as a Zen meditation hall, which is peace and designed to be suited for performing Zen.

Zen may be solely performed, or may also be performed in a group. Zen performed in a group causes unity between the members joining Zen, and the effects by Zen is increased.

The server 20 is a computer connected to the plurality of terminal apparatuses 40 via communication network 18.

The communication network 18 connects the server 20 and the plurality of terminal apparatuses 40 to achieve data communication therebetween. The communication network 18 may be wired or wireless, or a combination thereof is also applicable. The communication network 18 may be publication communication network, network by a dedicated line, or network made up of a combination thereof. For example, the communication network is made up of one of telephone network, mobile communication network, dedicated communication line, local area network (LAN), wireless LAN, Bluetooth (registered trademark) or a combination thereof.

<Server>

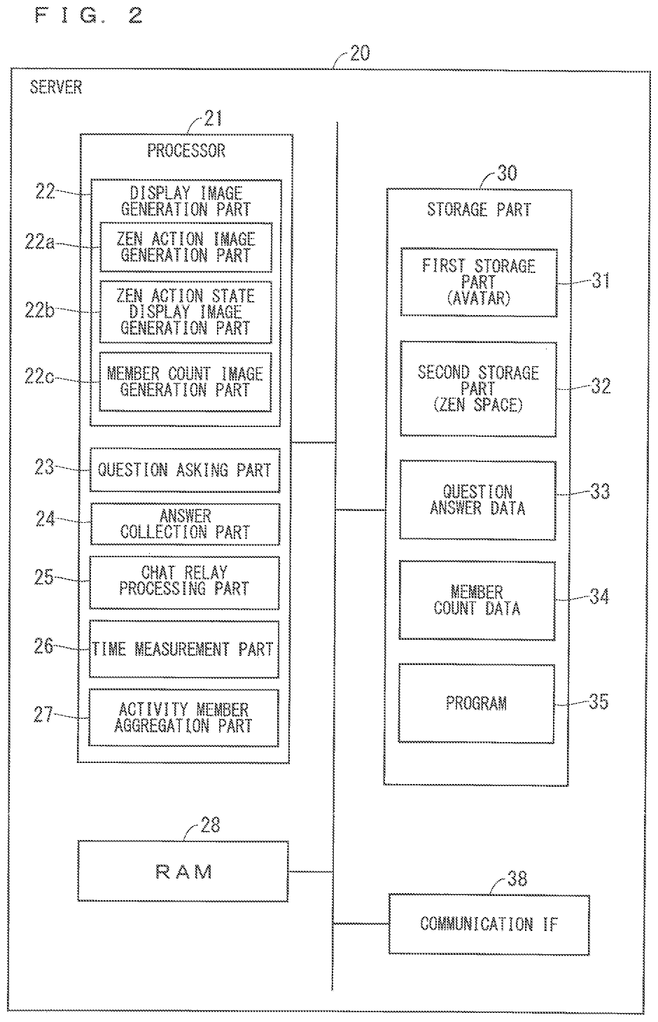

The server 20 is described more specifically. FIG. 2 is a block diagram of the server 20. The server 20 is made up of a computer, and executes an arithmetic operation in accordance with a program stored in the computer, thereby executing server processing. The server 20 may be one computer, or may also be a device in which a plurality of computers separately perform processing.

The server 20 includes a processor 21, a RAM 28, a storage part 30, and a communication interface 38, and they are connected via a bus line.

The processor 21 is made up of an arithmetic device executing arithmetic processing in accordance with a program, for example. A random access memory (RAM) is made up of a readable-writable memory, and is served as an operation region when the processor 21 performs processing. The storage part 30 is made up of a non-volatile storage medium such as a hard disk device or a flash memory. A program 35 is stored in the storage part 30. Processing at a time of performing arithmetic processing by the processor 21 is described in the program 35. Various types of data referenced when the processor 21 performs processing are stored in the storage part 30. The storage part 30 may be a single storage device, or may also be made up of a plurality of storage devices. A communication interface 38 is a communication circuit for the server 20 performing communication via the communication network 18. The server 20 can perform interactive communication with the plurality of terminal apparatuses 40 via the communication interface 38 and the communication network 18.

The processor 21 executes the arithmetic processing in accordance with the processing described in the program 35, thereby executing operations of a display image generation part 22, a question asking part 23, an answer collection part 24, a chat relay processing part 25, a time measurement part 26, and an activity member aggregation part 27. The processing of the display image generation part 22 includes processing as a Zen action image generation part 22a, a Zen action state display image generation part 22b, and a member count image generation part 22c. The display image generation part 22 generates a Zen action image in which some of the plurality of avatars perform Zen in the Zen space in accordance with an input regarding the Zen action performed by the members as the Zen action image generation part 22a. The display image generation part 22 may include a processing part as the Zen action state image generation part 22b and a member count image generation part 22c. The Zen action state display image generation part 22b is a processing part generating a Zen action state display image based on data acquired by a body state acquisition part 43 in the terminal apparatus 40. The member count image generation part 22c is a processing part generating an image in accordance with the counted number counted by the activity member aggregation part 27. The question asking part 23 provides a question after displaying the Zen action image, and the answer collection part 24 collects an answer corresponding to the question. The chat relay processing part 25 executes processing of relaying a chat between the plurality of terminal apparatuses 40. The time measurement part 26 performs processing of measuring a time at which the avatar performs Zen in the Zen space in accordance with the input regarding the Zen action from the members. The activity member aggregation part 27 executes processing of counting the number of members performing Zen at the same time in accordance with the input regarding the Zen action from the members. Each processing is further described in detail hereinafter.

The storage part 30 includes a first storage part 31 storing a plurality of avatars and a second storage part 32 storing a virtual Zen space. It is not necessary to previously store the avatar and the Zen space in the server 20 regardless of whether or not Zen is performed in the virtual Zen space. It is also applicable that at least one of the avatar and/or the Zen space is uploaded from the terminal apparatus 40 to the server 20 every time an owner (member) of the terminal apparatus 40 performs Zen.

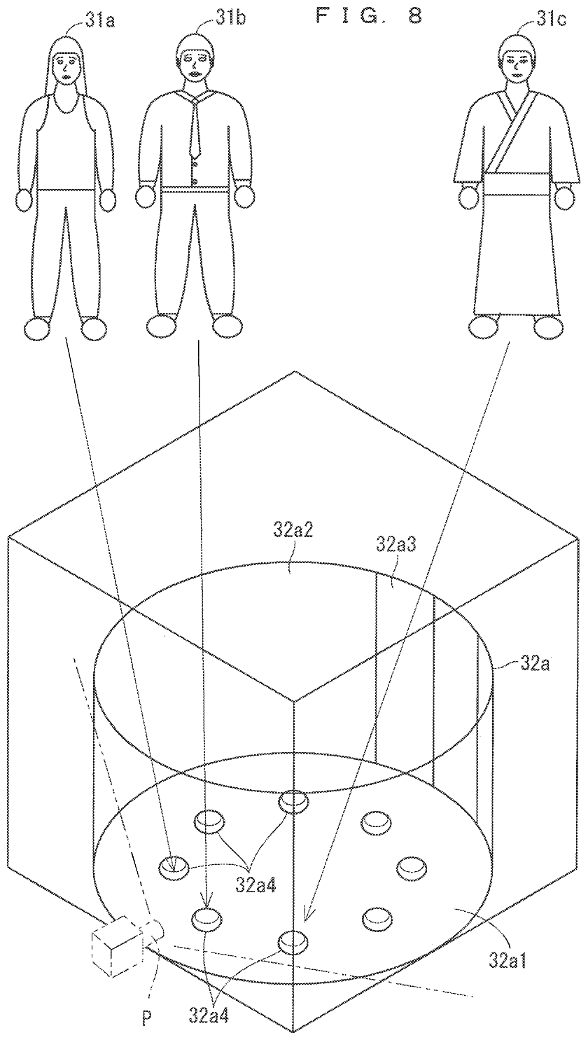

The avatar is alter ego data of the owner of the terminal apparatus 40 on a computer. The avatar may be personalized data. The avatar may be expressed by three-dimensional data or two-dimensional data. FIG. 8 referenced hereinafter illustrates an example that the avatar is the personalized three-dimensional data. The avatar may be created by the owner of the terminal apparatus 40. For example, it is applicable that candidates of the plurality of avatars are displayed on the terminal apparatus 40, and the owner of the terminal apparatus 40 selects and inputs one of them, thereby setting an avatar corresponding to the owner (member). At this time, the owner may individually select parts of the avatar, thereby creating the avatar. For example, candidates of a contour of a face, a hair style, eyes, a nose, a mouth, a body type, and clothes may be displayed as the parts on the terminal apparatus 40. The owner may select each part to create the avatar in accordance with a combination of the selected parts. The avatar may be created by any method regardless of the above description. A name, a nickname, or an identification number, for example, of the owner of the terminal apparatus 40 may be associated with the avatar.

As described above, the owner of the terminal apparatus 40 is the member belonging to the group. Thus, the first storage part 31 stores the plurality of avatars corresponding to the plurality of members belonging to the group. The plurality of avatars are associated with specific information of the plurality of members and stored in the first storage part 31.

The virtual Zen space is data of a Zen space having a surrounding landscape and brightness appropriate to perform Zen achieved not by an actual space but by expression which can be processed by a computer. The virtual Zen space may be data expressing a space (for example, a Zen meditation hall) where Zen is actually performed. The virtual Zen space may be data expressing a Zen space which is not existent but is considered to be ideal. The virtual Zen space may be expressed by three-dimensional data or two-dimensional data. FIG. 8 referenced hereinafter illustrates an example that the virtual Zen space is the three-dimensional data. The second storage part 32 may store only one virtual Zen space or a plurality of virtual Zen spaces. Described herein is an example that the second storage part 32 stores the plurality of virtual Zen spaces.

The processor 21 refers to the avatar and the virtual Zen space described above at a time of executing processing of generating Zen action image as the Zen action image generation part 22a described above.

The storage part 30 stores question answer data 33 and member count data 34. When the processor 21 executes processing as the question asking part 23 and the answer collection part 24, the question answer data 33 is generated and updated. When the processor 21 executes processing as the activity member aggregation part 27, the member count data 34 is generated and updated.

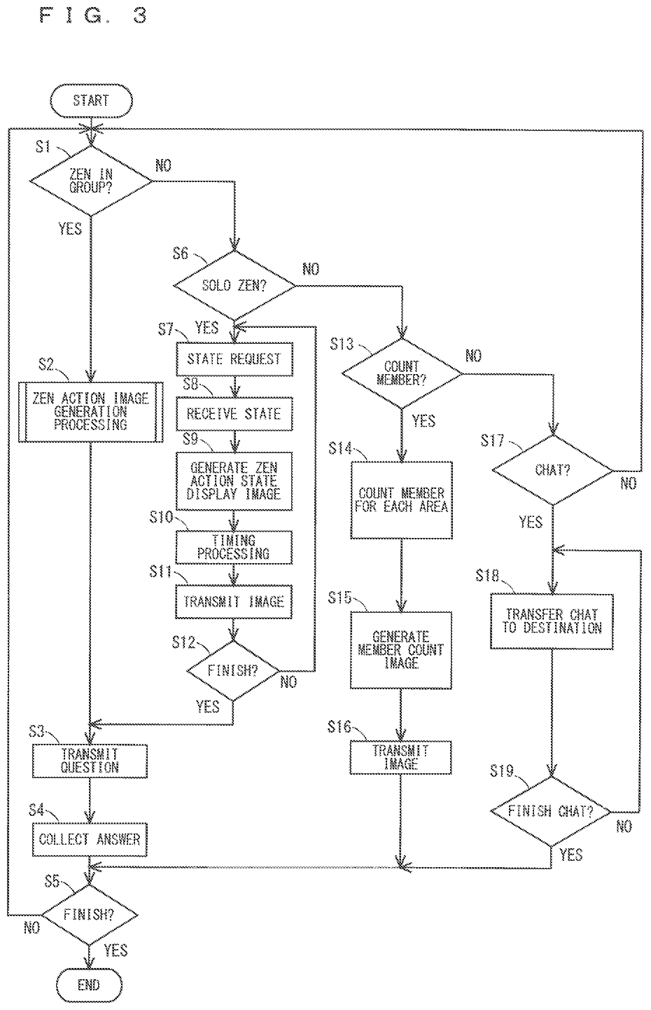

FIG. 3 is a flow chart illustrating a processing example of the server 20 based on the input from the terminal apparatus 40.

As illustrated in FIG. 3, in Step S1, the server 20 determines whether or not the member performs Zen in the group. That is to say, when a member owning the terminal apparatus 40 inputs any of performing Zen in a group, solely performing Zen, counting a participating member, or chatting, the input is transmitted to the server 20 via the communication network 18. Accordingly, it is determined whether a member owning the terminal apparatus 40 performs Zen in the group in accordance with the input of the member received in the server 20. Thus, each of the terminal apparatuses 40 functions as an input receiving part receiving the input from the member.

When it is determined that Zen is performed in Step S1, the processing proceed to Step S2. In Step S2, the server 20 performs processing of generating the Zen action image in accordance with the input regarding the Zen action from the user through the terminal apparatus 40. Herein, the Zen action image is an image in which some of the plurality of avatars corresponding to the plurality of members belonging to the group perform Zen in the Zen space, and the more specific example thereof is described hereinafter. This processing is described in more detail in relation to the processing with the terminal apparatus.

After Step S2, question data which has been previously set is transmitted to the terminal apparatus 40 through the communication network 18 in Step S3. Accordingly, a question sentence described in the question data is displayed in the terminal apparatus 40. When the user inputs an answer in the terminal apparatus 40, the answer is transmitted to the server 20 through the communication network 18.

In next Step S4, the server 20 collects the answer, and stores the answer as the question answer data 33 in the storage part 30.

In next Step S5, presence or absence of the action regarding Zen using the present system is determined based on the input transmitted from the terminal apparatus 40 through the communication network 18, and when it is determined that the action is finished, the processing is finished. When it is determined that the action is not finished but there is an action regarding the other Zen, the processing returns to Step S1.

When it is determined that Zen is not performed in a group in Step S1, the processing proceed to Step S6. It is determined whether or not Zen is solely performed in Step S6. The determination herein is performed based on the input of the user via the terminal apparatus 40 and the communication network 18 in the manner similar to Step S1 described above. When it is determined that Zen is solely performed, the processing proceeds to Step S7.

In Step S7, the server 20 transmits a request of a state of the user to the terminal apparatus 40 via the communication network 18. Accordingly, the terminal apparatus 40 acquires the state of the user, and transmits the acquired state of the user to the server 20 via the communication network 18. In next Step S8, the server 20 receives the state of the user.

In next Step S9, the server 20 generates the Zen action state display image. The Zen action state display image is an image of displaying the state of the user performing Zen. The state of the user performing Zen is a pulsation and breaths par minutes, for example. An example of the Zen action state display image is described hereinafter.

In Step S10, the server 20 executes timing processing regarding the time at which Zen is performed. For example, a time elapsed after Zen is started is measured. A start time of Zen may be a time when an input of solely performing Zen is transmitted from the terminal apparatus 40, or a time after an elapse of a predetermined period of time which has been previously set since the input is transmitted.

In next Step S11, the server 20 transmits the Zen action state display image to the terminal apparatus 40 which has input that Zen is solely performed via the communication network 18. Accordingly, Zen action state display image is displayed in the terminal apparatus 40.

In next Step S12, it is determined whether or not Zen solely performed is finished. The finish may be determined by whether or not the time elapsed after Zen is started described above is longer than the predetermined period of time which has been previously set. The finish may be determined when the user inputs that Zen is finished via the terminal apparatus 40. When it is determined that Zen is not finished, the process returns to Step S7, and the processing described above is repeated. When it is determined that Zen is finished, the processing proceeds to Step S3.

When it is determined that Zen is not solely performed in Step S6, the processing proceeds to Step S13.

In Step S13, the server 20 determines whether or not the member is counted based on the input transmitted from the terminal apparatus 40 via the communication network 18. When it is determined that the member is counted, the processing proceeds to Step S14.

In Step S14, the server 20 counts the number of the members performing Zen using the present system at that time based on the input regarding the Zen action in the plurality of terminal apparatuses 40 connected to the server 20 via the communication network 18. For example, the number of members may be counted for each area. The area may be continents or countries in the world based on positions where the terminal apparatuses 40 performed by the users are actually located.

In next Step S15, the server 20 generates a member count image expressing the counted number of members.

In next Step S16, the server 20 transmits the member count image to the terminal apparatus 40 to which the member count is inputted via the communication network 18. Subsequently, the processing proceeds to Step S5.

When it is determined that the member is not counted in Step S13 described above, the processing proceeds to Step S17.

In Step S17, the server 20 determines whether or not the chat is performed based on the input transmitted from the terminal apparatus 40 via the communication network 18. When it is determined that the chat is performed, the processing proceeds to Step S18, and when it is determined that the chat is not performed, the processing returns to Step S1.

In Step S18, the server 20 transfers chat data received from the terminal apparatus 40 via the communication network 18 to a destination (the terminal apparatus 40) of the chat data via the communication network 18.

In next Step S19, it is determined whether or not the chat is finished. For example, when finish of chat is inputted on a side of the terminal apparatus 40, the input thereof is transmitted to the server 20 via the communication network 18, and the finish of the chat is determined. When the user continues the chat, the processing returns to Step S18, and transfer processing of the chat is repeated between the users in which the chat is established. Accordingly, chat information is displayed in each of the plurality of terminal apparatuses 40 performing the chat. When the chat is finished, the processing proceeds to Step S5.

<Terminal Apparatus>

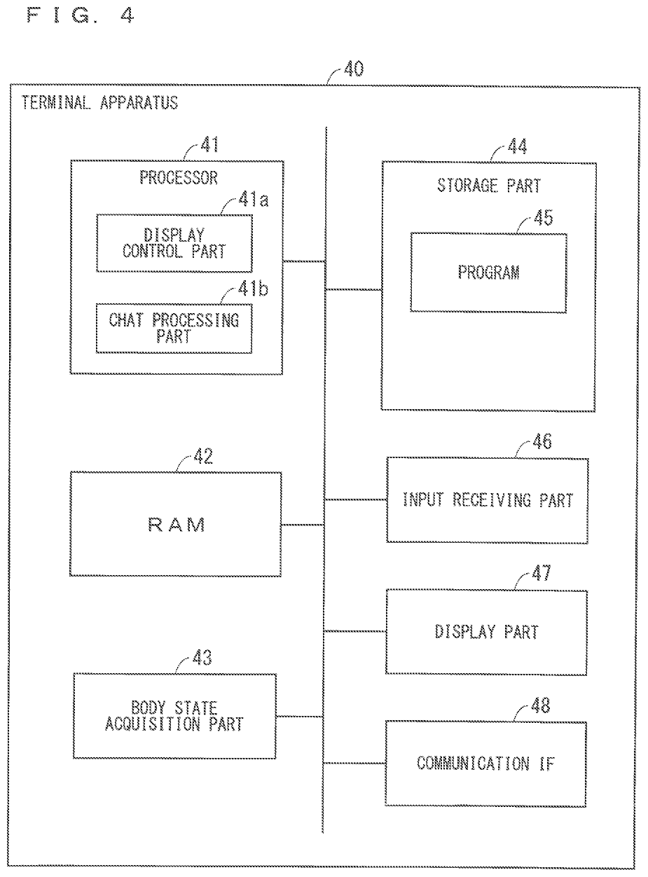

The terminal apparatus 40 is described more specifically. FIG. 4 is a block diagram of the terminal apparatus 40. The terminal apparatus 40 is made up of a computer, and executes an arithmetic operation in accordance with a program stored in the computer, thereby executing client processing. Each of the plurality of members belonging to the group described above owns the terminal apparatus 40. The plurality of members perform Zen using the terminal apparatuses 40.

The terminal apparatus 40 includes a processor 41, a RAM 42, the body state acquisition part 43, a storage part 44, an input receiving part 46, a display part 47, and the communication interface 48, and they are connected via a bus line.

The processor 41 is made up of an arithmetic device executing arithmetic processing in accordance with a program in the manner similar to the processor 21. The RAM is served as an operation region when the processor 41 performs processing. The storage part 44 is made up of a non-volatile storage medium such as a flash memory or a hard disk device. The storage part 44 stores a program 45. Processing for the processor 41 performing the arithmetic processing is described in the program 45. The communication interface 48 is a communication circuit for communication of the terminal apparatus 40 with the server 20 via the communication network 18. Each of the plurality of terminal apparatuses 40 can perform interactive communication with the server 20 via the communication interface 48 and the communication network 18.

The input receiving part 46 is a man-machine interface receiving input from the member owning the terminal apparatus 40. The input receiving part 46 particularly receives an input regarding the Zen action from the member. The input receiving part 46 may be a touch panel incorporated into a display screen of a smartphone or a tablet computer, for example. Alternatively, the input receiving part 46 may be a switch. The input receiving part may determine a movement of the user based on a camera and an image taken by the camera to receive the input. For example, it is applicable that an image of a portion of the user is extracted from the image taken by the camera and pattern matching processing is performed on the extracted image to determine whether or not the user performs Zen. In this case, when the user enters a state of performing Zen, start of Zen is inputted, and when the state where the user performs Zen is not determined from the state where the user performs Zen, finish of Zen may be inputted.

The display part 47 is a liquid crystal display device or an organic electro-luminescence (EL) display device, for example, and is a display device displaying various types of information. In the present embodiment, the display part 47 displays the image (particularly, the Zen action image) generated in the display image generation part 22 and transmitted from the server 20.

The body state acquisition part 43 is a device acquiring a body state such as a pulsation and breaths par minutes, for example, of the member. The body state acquisition part 43 may acquire the body state by analyzing a body surface image acquired by an imaging element, or may acquire the body state by analyzing a body surface state of the member based on output of a motion sensor such as a Doppler sensor, for example. The body state acquisition part 43 may be a part acquiring the body state such as a pressure sensor detecting the body surface state. The sensor included in the body state acquisition part 43 may be integrally incorporated into the terminal apparatus 40, or may also be connected to the terminal apparatus 40 via Bluetooth (registered trademark) communication. For example, it is applicable that the body state acquisition part 43 has a bracelet form into which a sensor detecting a pulsation is incorporated, and is attached to an arm of the member to be connected to the terminal apparatus 40 via radio communication such as Bluetooth (registered trademark) communication.

The storage part 44 stores the program 45, and the processor 41 executes arithmetic processing in accordance with processing described in the program 45, thereby executing an operation as a display control part 41a and a chat processing part 41b. The display control part 41a is a processing part performing display control in the display part 47. In the present embodiment, the display control part 41a executes control of displaying the image transmitted from the server 20 on the display part 47. When the chat processing part 41b receives an input of conversion data from the plurality of members via the input receiving part 46 (including the input receiving part 46 of the terminal apparatus 40 of a subject member) from the plurality of terminal apparatuses 40 corresponding to the plurality of members, the chat processing part 41b executes processing of displaying a conversion of the plurality of members in a chat form based on the conversion data. Receipt of the conversion data includes data through the input receiving part 46 in the terminal apparatus 40 of the subject member and data through the input receiving part 46 and the communication network 18 in the other terminal apparatus 40. Thus, the conversion of the subject member and the conversation of the other member are expressed in an order of statement while the member which has made a statement is distinguished. It is also applicable that the program 45 is stored in a storage medium in a server providing a program, and is downloaded from the server to the terminal apparatus 40 via communication network in response to a request from the terminal apparatus 40 to be installed in the terminal apparatus 40. In this case, the program 45 is stored in the storage part 44 to be able to be executed in the terminal apparatus 40 while being installed in the terminal apparatus 40.

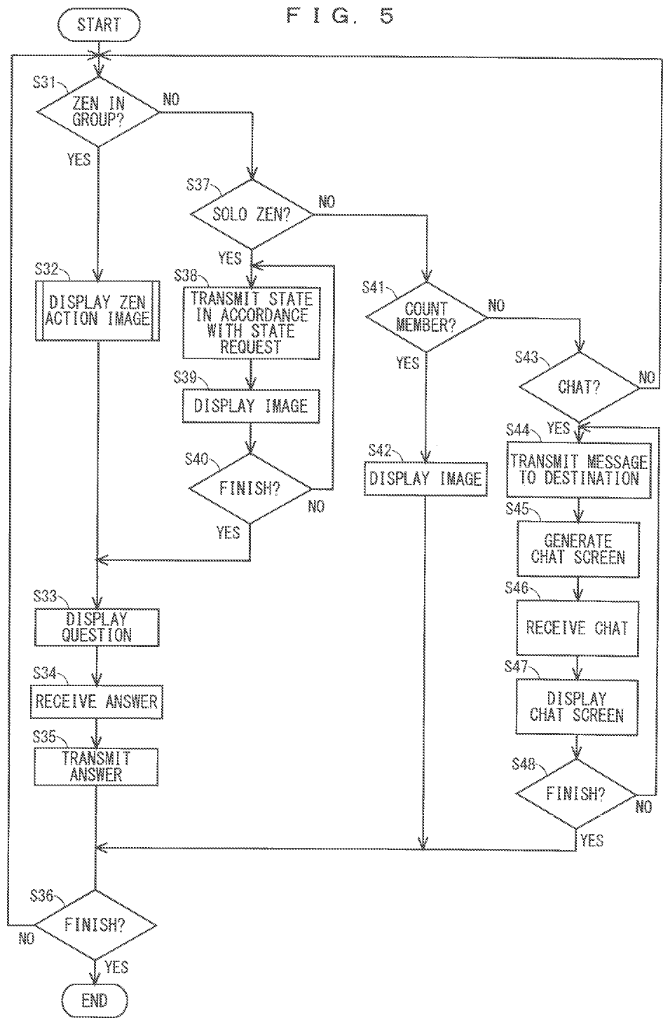

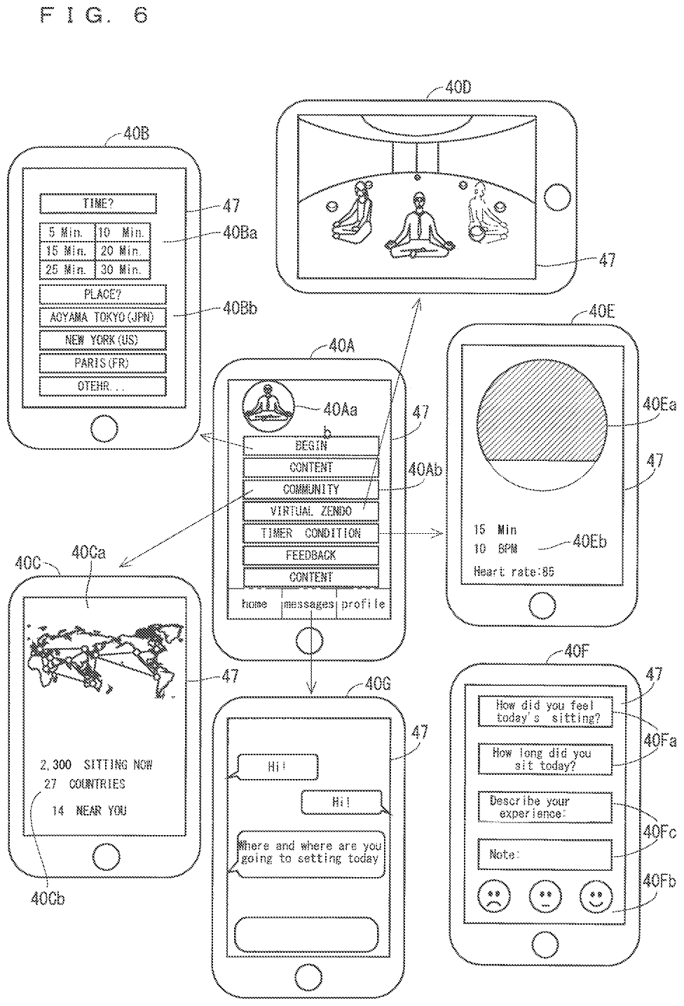

FIG. 5 is a flow chart illustrating a processing example in the terminal apparatus 40. FIG. 6 is an explanation diagram illustrating a transition example of the screen display of the terminal apparatus 40. In FIG. 6, the terminal apparatuses 40 having different types of screen display are distinguished by assigning A-G to a reference sign 40.

An avatar representative image 40Aa and a selection image 40Ab are displayed on an initial screen of the terminal apparatus 40A. The avatar image is an image indicating an avatar of the subject member. The selection image 40Ab is an image for inputting a structure regarding Zen. For example, the selection image 40Ab is an image for inputting whether Zen is performed in a group, Zen is solely performed, a member performing Zen is displayed, or a chat is performed. In the present example, a display image of “BEGIN” is an image for inputting an initial setting regarding Zen, a display image of “COMMUNITY” is an image for inputting that the member performing Zen is displayed, the display image of “VIRTUALZENDO” is an image for inputting that Zen is performed in a group, and the display image of “TIMER CONDITION” is an image for inputting that Zen is solely performed. The display image of “TIMER CONDITION” is also an image for inputting that the body state is acquired. “Home” image, “message” image, and “profile” image may be displayed by swiping a lower portion of the screen from a lower side to an upper side. The “message” image is an example of an image for inputting that a chat is performed. “CONTENT” image for displaying a video for guiding how to perform Zen or “FEEDBACK” image for posting an opinion may be displayed, for example. “Profile” described above may be a display for registering or changing a history of the subject member.

When the display image of “BEGIN” is tapped, a terminal apparatus 40B enters a state of displaying a setting screen, for example. The setting screen may include a screen 40Ba for inputting a time of performing Zen or a screen 40Bb for inputting a location of a virtual Zen space, for example. Herein, a plurality of virtual Zen spaces are associated with locations (herein, cities in the world such as Aoyama, Tokyo (Japan) or New York (US)). The member selects an area, thereby being able to select one of the plurality of Zen spaces to perform Zen. It is also applicable that the plurality of virtual Zen spaces are not associated with the locations but are associated with design types of the Zen spaces (a wood-grain pattern or a brick-like pattern, for example), for example.

When the display image of “COMMUNITY” is tapped, a terminal apparatus 40C displays the number of members performing Zen. For example, displayed are an image 40Ca locations where Zen is performed in a world map are displayed and an image 40Cb indicating the number of members performing Zen in the whole world, the number of areas (number of countries) where Zen is performed, and the number of members where Zen is performed near the subject member. The terminal apparatus 40 can specify a location of the terminal apparatus 40 itself using a GPS sensor mounted to the terminal apparatus 40 itself or a Wi-Fi (registered trademark) access point as a connection destination, for example, and positional information of the terminal apparatus 40 is also transmitted to the server 20. Accordingly, the server 20 can recognize a position of each connected terminal apparatus 40, and specifies the number of the terminal apparatuses 40 of the members performing Zen for each location.

When the display image of “VIRTUAL ZENDO” is tapped, for example, a terminal apparatus 40D displays an image in which an avatar performs Zen in the Zen space. When the display image of “TIMER CONDITION” is tapped, for example, a terminal apparatus 40E displays an image 40Ea indicating an elapsed time with regard to a scheduled time of performing Zen (in FIG. 6, a colored region in a circle indicating the scheduled time indicates the elapsed time) and an image 40Eb indicating the scheduled time, breaths par minutes (BPM), or a heart rate, for example.

A terminal apparatus 40F displays an image indicating a question, for example. For example, displayed are a question sentence 40Fa, an image 40Fb expressing an answer of the question by options, and an image 40Fc for inputting a special note by a sentence.

When the “message” image is tapped, for example, a terminal apparatus 40G displays a chat image in which contents of each conversation are distinguished for each member who has made a statement.

A processing example in the terminal apparatus 40 is described. In Step S31, the terminal apparatus 40 determines whether or not the member performs Zen in a group. For example, the action that the user owning the terminal apparatus 40 taps the display image of “VIRTUAL ZENDO” in an initial setting screen of the terminal apparatus 40A (refer to the terminal apparatus 40A) indicates that the user inputs that the user performs Zen in a group. In this manner, the terminal apparatus 40 functions as an input setting part receiving the input from the member. When the determination is YES in Step S31, the processing proceeds to Step S32. The input in the terminal apparatus 40 is transmitted to the server 20 via the communication network 18 together with specific information of the member.

In Step S32, the terminal apparatus 40 performs processing for displaying the Zen action image (refer to the terminal apparatus 40D) transmitted from the server 20. This processing is described in more detail in relation to the processing with the server 20.

After Step S32, in Step S33, the terminal apparatus 40 executes processing of displaying an image including a question sentence (refer to the terminal apparatus 40F) on the display part 47 based on question data transmitted from the server 20. In next Step S34, the terminal apparatus 40 receives an answer of the question through the input receiving part 46. In next Step S35, the received answer is transmitted to the server 20.

In next Step S36, presence or absence of the Zen action using the present system is determined based on the input to the input receiving part 46. For example, when the user taps the “home” image to return the screen of the terminal apparatus 40 to a home screen or turns off the terminal apparatus 40, the determination is YES, and the processing is finished. When it is determined that Zen action is not finished, the processing returns to Step S31.

When it is determined that Zen is not performed in a group in Step S31, the processing proceeds to next Step S37.

It is determined whether or not Zen is solely performed in Step S37. When it is determined that Zen is solely performed through the input receiving part 46, the processing proceeds to Step S38. For example, when the display image of “TIMER CONDITION” is tapped in the initial setting screen, the processing proceeds to Step S38. This input is also transmitted to the server 20.

In Step S38, the body state of the user is acquired through the body state acquisition part 43 in response to the request from the server 20, and body state data is transmitted to the server 20. In next Step S39, the image indicating the body state (refer to the terminal apparatus 40E) is displayed on the display part 47 based on the image transmitted from the server 20. In next Step S40, it is determined whether or not Zen solely performed is finished. For example, it is applicable that a time after Zen is started is measured and when the time passes a preset scheduled time of performing Zen, Zen is finished. It is also applicable that when the image of selecting finish is displayed on the screen and the image is tapped, it is determined that Zen is finished. When it is determined that Zen solely performed is not finished, the processing returns to Step S38, and the processing described above is repeated. It is not necessary to acquire the body state and display the body state. In this case, it is also applicable that the time is simply measured and the elapsed time is displayed or it is displayed that Zen is finished when a scheduled time passes. When it is determined that Zen solely performed is finished, the processing proceeds to Step S33.

When it is determined that Zen is not solely performed in Step S37, the processing proceeds to Step S41. In Step S41, it is determined whether or not the member is counted. For example, when the display image of “COMMUNITY” is tapped in the initial setting screen, YES is determined. In this case, the terminal apparatus 40 transmits a command of counting the member to the server 20. Accordingly, the server 20 counts the member performing Zen, generates the member count image expressing the number of counted members, and transmits the image to the terminal apparatus 40 via the communication network 18. Accordingly, as illustrated in Step S42, the terminal apparatus 40 displays the image expressing the member on the display part 47 (refer to the terminal apparatus 40C). Subsequently, the processing proceeds to Step S36.

When it is determined that the member is not counted in Step S41, the processing proceeds to Step S43.

In Step S43, the terminal apparatus 40 determines whether or not the chat is performed based on the input via the input receiving part 46. For example, when the “message” image is tapped in the initial setting screen, it is determined that the chat is performed. When the determination is NO, the processing returns to Step S31, and when the determination is YES, the processing proceeds to Step S44.

In Step S44, an address and message data inputted via the input receiving part 46 is transmitted to the server 20 via the communication network 18. In next Step S45, the terminal apparatus 40 generates a chat image including the transmitted message, and displays the chat image on the display part 47. In next Step S46, the message from the other member as the address is received. In next Step S47, a chat screen including the message of the subject member and the message from the other member as the address is displayed on the display part 47 (refer to the terminal apparatus 40G). In next Step S48, presence or absence of finish of the chat is determined. For example, when input of selecting finish of the chat is performed or a command of transition to the other screen is transmitted, it may be determined that the chat is finished. When the determination is NO, the processing returns to Step S44, and the processing of displaying the subsequent chat is executed. When the determination is YES, the processing proceeds to Step S36.

<Zen Action Image Display Processing in Group>

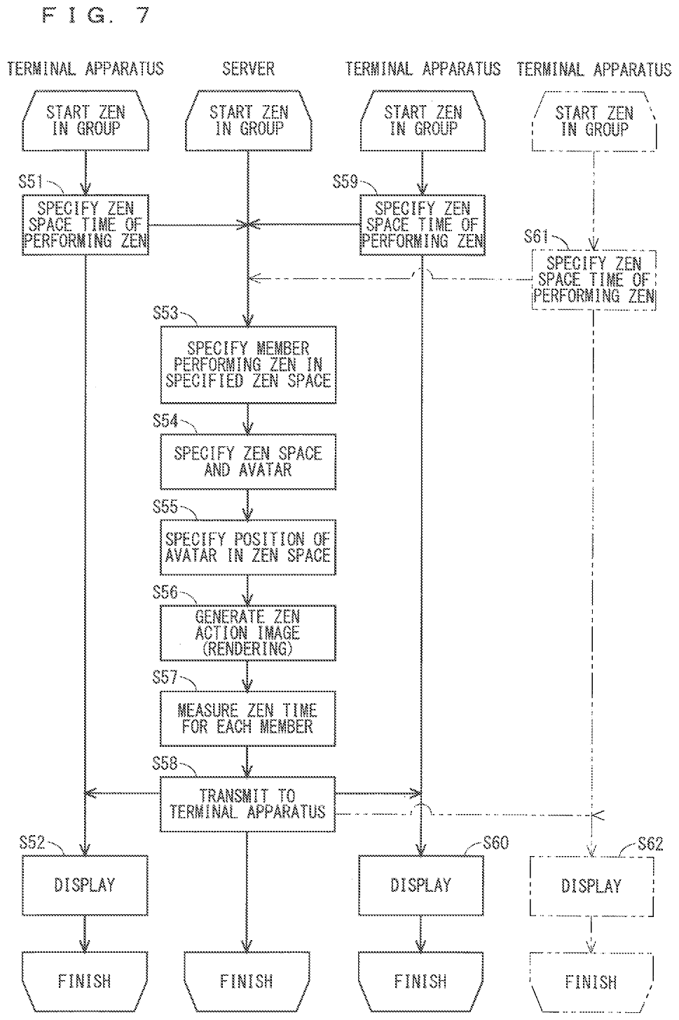

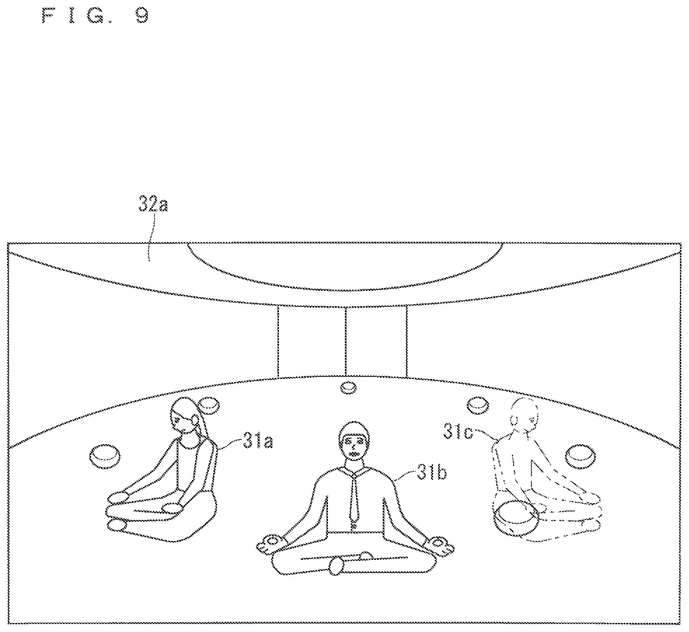

Described is processing of displaying a Zen action image in a group. FIG. 7 is a flow chart illustrating processing for generating and displaying the Zen action image. FIG. 8 is a diagram illustrating an example of avatars 31a, 31b, and 31c and a Zen space 32a. FIG. 9 is a diagram illustrating an example of the Zen action image.

The first storage part 31 stores the plurality of avatars 31a, 31b, and 31c. The avatars 31a and 31b are associated with the members belonging to the group, respectively. The avatar may be associated with a master avatar as a coach in the members belonging to the group. The avatars 31a, 31b, and 31c may be personalized three-dimensional data, for example. The avatars 31a, 31b, and 31c may be two-dimensional data.

The second storage part 32 stores the Zen space 32a. The Zen space 32a is data expressing a Zen space appropriate to perform Zen, and is three-dimensional data, for example. The second storage part 32 may store a plurality of Zen spaces. The plurality of Zen spaces may be associated with a plurality of virtual Zen spaces set in the terminal apparatus 40B, for example. The Zen space 32a may be expressed by two-dimensional data. For example, in FIG. 8, the Zen space 32a is expressed by a circular plate-like floor 32al, a wall 32a2 defining a circle provided around the floor 32al, a door 32a3 provided in a part of the wall 32a2, and a plurality of floor cushions 32a4 provided at regular intervals along a circumferential direction of an inner side of an outer periphery of the floor 32al. Alternatively, the Zen space may be expressed as a room having rectangular parallelepiped shape or a natural space.

When it is determined that the avatars 31a and 31b perform Zen in the Zen space 32a, each of the avatars 31a and 31b is set in a position corresponding to one of the floor cushions 32a4. A posture of each of the avatars 31a and 31b is set to be directed to the wall 32a2 and sit on the floor 32al. Thus, the server 20 executes rendering processing so that an image in which the avatars 31a and 31b each disposed in a posture of sitting on the floor cushion 32a4 in the Zen space 32a can be observed from a predetermined viewpoint position P is converted into a two-dimensional image, thereby being able to generate the Zen action image in which the avatars 31a and 31b perform Zen in the Zen space 32a (refer to FIG. 9). The Zen action image may be a still image or a video. The viewpoint position P may be a position where an image of all the floor cushions 32a4 can be taken (that is to say, a position where an image of all the avatars can be taken) in the Zen space 32a from a front side position of the avatars 31a and 31b corresponding to the users, for example. It is also applicable that the user swipes the display part 47 to transmit an operation of changing the viewpoint position P from the terminal apparatus 40 to the server 20. The server 20 may generate the Zen action image in accordance with the changed viewpoint position P. A master avatar 31c performing Zen may be expressed in the Zen action image (refer to a dashed-two dotted line in FIG. 9). A name or a nickname of the member may be displayed on each of the avatars 31a and 31b. A detailed profile of the member corresponding to each of the avatars 31a and 31b may be displayed by selecting and tapping each of the avatars 31a and 31b.

When the Zen space 32a and the avatars 31a and 31b are three-dimensional data and the Zen action image is generated based on these types of data, the Zen action image rich in a sense of reality can be generated. The Zen action image viewed from various direction can be easily generated. Furthermore, the Zen action image in which the avatars 31a and 31b perform various types of operation can be easily generated.

When the Zen space 32a and the avatars 31a and 31b are two-dimensional data, the Zen action image may be generated by overlapping the avatars 31a and 31b with the Zen space 32a.

In generating and displaying the Zen action image described above, as indicated in Steps S51 and S59, specific information of the Zen space where Zen is performed and a scheduled time of performing Zen are transmitted from the plurality of terminal apparatuses 40 to the server 20. The Zen space where Zen is performed and the time of performing Zen are set through the setting screen in the terminal apparatus 40B, for example.

Subsequently, in Step S53 in the server 20, the member performing Zen in the specified Zen space is specified based on the input from each terminal apparatus 40. In next Step S54, the server 20 refers to the first storage part 31 and the second storage part 32 to specify the data expressing the Zen space and the avatars 31a and 31b corresponding to the members. In next Step S55, the positions of the avatars 31a and 31b in the Zen space 32a are specified. In next Step S56, the avatars 31a and 31b are disposed in predetermined postures in the Zen space 32a as the three-dimensional data to generate the Zen action image (two-dimensional image) which can be observed from a predetermined viewpoint position. In next Step S57, a time of performing Zen may be measured for each member. The time measurement may be started at the other timing such as a timing at which the terminal apparatus 40 transmits that Zen is performed to the server 20, for example.

In next Step S58, the generated Zen action image is transmitted to each terminal apparatus 40. Accordingly, the Zen action image is displayed in each terminal apparatus 40 as indicated in Steps S52 and S60. Displayed in the present system are the plurality of avatars 31a and 31b performing Zen in the same Zen space 32a, however, when one member performs Zen in a Zen space, one avatar performing Zen in the Zen space may be displayed.

The processing described above is repeated until Zen performed in a group is finished. In the middle of performing Zen, the avatars 31a and 31b are not basically moved but are kept in a still state. It is determined that Zen is finished when a preset scheduled time of Zen has passed or an optional input of finishing Zen is performed on all the terminal apparatuses 40, for example.

When the terminal apparatus 40 of the other member inputs that Zen is performed in the Zen space as indicated in Step S61 in the middle of performing Zen, an avatar corresponding to a member halfway participating in Zen may be added to the subsequent Zen action image. Accordingly, the Zen action image in which the avatar is newly added to the existing avatars is displayed in the terminal apparatuses 40 of the members who have already participated and the terminal apparatus 40 of the member who has newly participated (refer to Steps S52, S60, and S62). Some member may finish Zen in the middle of performing Zen in some cases. Examples thereof include a case where a preset scheduled time has passed earlier or an optional input of finishing Zen is performed one some member. In this case, the avatar corresponding to such a member may not be displayed in the Zen action image.

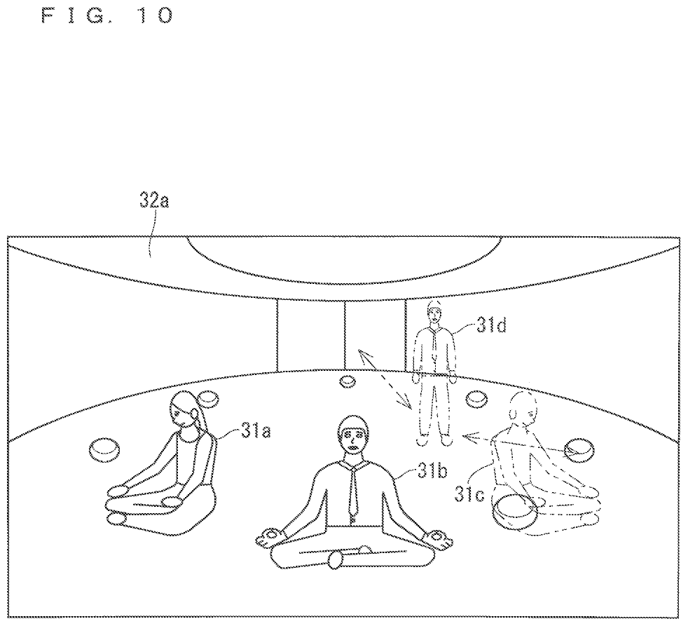

As illustrated in FIG. 10, at least one of the action of the avatar 31d starting Zen in the Zen space 32a and/or the action thereof finishing Zen therein may be displayed in the beginning, the end, or the middle of generation of the Zen action image. For example, the avatar 31d moving from a position of a door 32a3 toward the floor cushion 32a4 and starting sitting or in contrast, the avatar 31d standing from a state of performing Zen and moving toward the door may be displayed as a continuous still image or a video. Such an image may be generated by performing rendering processing while the avatar 31d is moved in accordance with a preset movement pattern in the Zen space 32a, for example.

<Outline>

According to the Zen facilitation system 10 having such a configuration, the member can visually recognize the plurality of avatars 31a and 31b performing Zen in the virtual Zen space 32a through the display part 47. When Zen is performed with half-closed eyes, the Zen action image can be visually recognized in the middle of performing Zen. The visual recognition may be performed immediately before performing Zen. Accordingly, the member can perform Zen in an actual space while imagining a state where the member performs Zen in the Zen space with the other member in his/her head. Accordingly, assigned is a simulated experience in which the member performs Zen with the other member in the Zen space which is peace and designed to be suited for performing Zen even when the member cannot go to the Zen space due to a positional, temporal, or epidemiological limitation. Accordingly, a mental activity by Zen is facilitated.

When the master avatar 31c is displayed in the Zen action image, the member can perform Zen in the actual space while having a sense of unity to perform Zen with the master in the Zen space 32a, thus a mental activity by Zen is further facilitated.

The present Zen facilitation system 10 includes the plurality of terminal apparatuses 40 including the input receiving part 46 and the display part 47, thus each member can perform an input regarding the Zen action, and can further see his/her avatar performing Zen in the virtual Zen space together with the other avatar through the terminal apparatus 40.

The plurality of terminal apparatuses 40 and the server 20 are connected via the communication network 18, thus the server 20 can perform communication with the plurality of terminal apparatuses 40 to execute adjustment processing between the plurality of terminal apparatuses 40 such as specific processing of specifying the avatars 31a and 31b performing Zen in a predetermined Zen space, for example.

The processing of the display image generation part 22 needs not be performed in the server 20, but may be performed in the terminal apparatus 40 within an achievable range, or may also be separately performed between the server 20 and the terminal apparatus 40.

The question is displayed on the display part 47 after the Zen action image is displayed, and the answer inputted through the input receiving part 46 is collected. Thus, the question can be provided after the member performs Zen, and the answer can be collected. Self-observation is facilitated by performing answering, and self-observation is also facilitated by thinking about passed contents of Zen.

The input of the conversation data is received from the plurality of terminal apparatuses 40, and the conversation between the members is displayed in the chat form based on the conversation data, thus the communication between the members is facilitated. For example, it is also applicable that the participating member is displayed in a list form, and the chat with the member in the list as the destination can be performed.

The body state of the member is acquired by the body state acquisition part 43, and the Zen action state display image based on the body state is displayed on the display part 47, thus the body state in performing Zen is easily grasped by the member. A degree of mental concentration may be estimated from the body state and displayed. In the present embodiment, the Zen action state display image is displayed when Zen is solely performed, however, the Zen action image of performing Zen in the group and the Zen action state display image may be displayed at the same time.

The server 20 includes a time measurement part 26, thus can grasp the time of performing Zen. Accordingly, finish of Zen can be determined after the predetermined scheduled time is finished. A history of performing Zen can be managed. The terminal apparatus 40 may include the time measurement part.

The activity member aggregation part 27 counts the number of members performing Zen at the same time in accordance with input through the input receiving part 46, and the image corresponding to the counted number is generated and displayed on the display part 47. Thus, the member can perform Zen while grasping the number of members performing Zen at the same time. Accordingly, the member can perform Zen while having a sense of unity with more members, thus concentration power can be further facilitated.

The second storage part 32 stores the plurality of Zen spaces, and the display image generation part 22 generates the Zen action image of the avatars 31a and 31b performing Zen in one Zen space selected from the plurality of Zen spaces. Thus, the member can see the image of his/her avatar performing Zen in the Zen space, which is considered to be appropriate for him/her to perform Zen, and the Zen action can be further facilitated.

The display image generation part 22 generates the Zen action image performing at least one of the action of the avatars 31a and 31b starting Zen in the Zen space 32a and/or the action thereof finishing Zen therein in accordance with the input regarding the Zen action through the input receiving part 46. Thus, the member can see a start action or a finish action of Zen of his/her avatar, thereby being able to easily switch his/her mind before and after performing Zen.

The Zen action image includes the image of the avatars 31a and 31b performing Zen in the still state in the Zen space 32a, thus can easily imagine the state of performing Zen in the Zen space 32a.

The server 20 receives the input regarding the Zen action via the terminal apparatus 40 and the communication network 18, generates the data expressing the Zen action image of the plurality of avatars 31a and 31b in the plurality of avatars, which correspond to the plurality of members belonging to the group, performing Zen in the virtual Zen space 32a in response to the receipt of the input, and transmits the data expressing the Zen action image to at least one of the plurality of terminal apparatuses 40. The data expressing the Zen action image of performing Zen in the virtual Zen space 32a may be a Zen action image converted into the two-dimensional image described above, or three-dimensional data in which the avatars 31a and 31b as three-dimensional data are disposed in the three-dimensional Zen space 32a.

In the former case, a load for the image processing can be reduced in the terminal apparatus 40. An amount of data transmitted to the terminal apparatus 40 can also be reduced. Thus, the former case is effective in generating the Zen action image based on the Zen space 32a as the three-dimensional data and the avatars 31a and 31b as the three-dimensional data. In the latter case, rendering processing appropriate to the display from the three-dimensional data to the display part 47 may be executed in the terminal apparatus 40.

The data expressing the Zen action image of performing Zen in the virtual Zen space 32a described above is transmitted to the plurality of terminal apparatuses 40 corresponding to the member performing Zen in the Zen space 32a. Thus, the plurality of members can see the avatars 31a and 31b performing Zen in the same Zen action image, and a sense of unity between the members is increased in performing Zen.

The server 20 receives processing of designating one of the plurality of Zen spaces via the terminal apparatus 40 and the communication network 18, and generates the data expressing the Zen action image of performing Zen in the designated one Zen space. Thus, the member can see the image of his/her avatar performing Zen in one Zen space selected from the plurality of Zen spaces. In this case, when the other member designates the same Zen space, the avatar corresponding to the other member and performing Zen in the same Zen space can also be observed.

When the terminal apparatus 40 receives the input regarding the Zen action, displayed on the display part 47 is the Zen action image of the plurality of avatars 31a and 31b in the plurality of avatars, which correspond to the plurality of members belonging to the group, performing Zen in the virtual Zen space 32a, thus the member owning the terminal apparatus 40 can visually recognize the Zen action image easily.

The terminal apparatus 40 includes the body state acquisition part 43 and displays the Zen action state image based on the data acquired by the body state acquisition part 43 on the display part 47, the body state of the member owning the terminal apparatus 40 at the time of performing Zen can be easily grasped.

The terminal apparatus 40 displays the image corresponding to the number of the members performing Zen at the same time on the display part 47, thus the member can easily grasp the number of members performing Zen at the same time.

### Second Embodiment

Each processing example in the server 20 and the terminal apparatus 40 described in the first embodiment is an example, thus it is not necessary to separate each processing in the present system 10 such as the processing in the server 20 and the terminal apparatus 40 in the first embodiment.

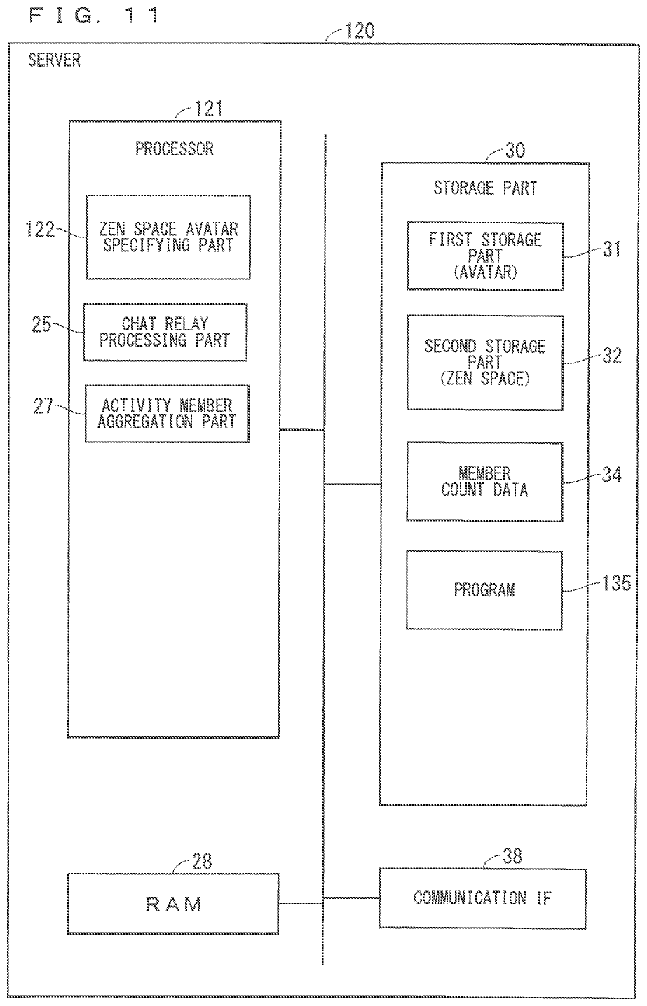

FIG. 11 is a block diagram of the server 120 corresponding to the server 20. As illustrated in the server 120, the display image generation part 22, the question asking part 23, the answer collection part 24, and the time measurement part 26 may be omitted in the server 20 illustrated in FIG. 2. The question answer data 33 may be omitted in the storage part 30. A processor 121 of the server 120 executes arithmetic processing in accordance with processing described in a program 135, thereby executing processing as the chat relay processing part 25 and the activity member aggregation part 27, and further executing processing as a Zen space avatar specifying part 122. The Zen space avatar specifying part 122 is a part executing processing of specifying the avatar performing Zen in a specified Zen space based on transmission data from each terminal apparatus 40 in the processing as the Zen action image generation part 22a described above. In the present processing, for example, when performing Zen in a group in any Zen space is inputted from any terminal apparatus 40, the avatar corresponding to one or the plurality of members performing Zen in the specified Zen space is specified, and the data regarding the specified Zen space and the specified avatar is transmitted to the terminal apparatus 40 of the member performing Zen in the Zen space. The data regarding the specified Zen space and the specified avatar may be identification data for specifying the Zen space and the avatar or may also be data itself expressing the Zen space and the avatar.

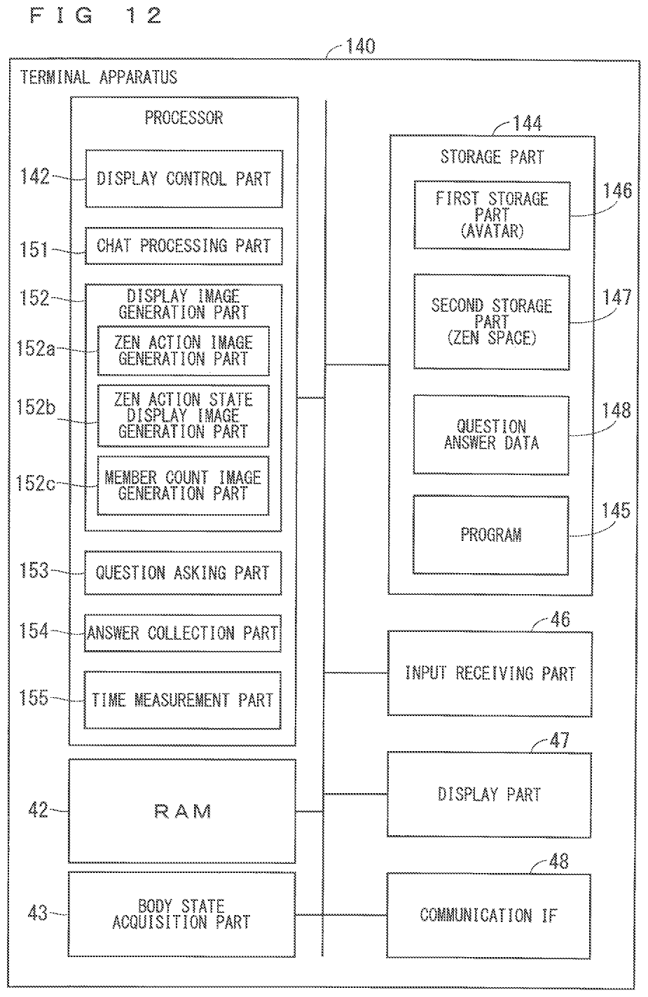

FIG. 12 is a block diagram of a terminal apparatus 140 corresponding to the terminal apparatus 40. As illustrated in FIG. 12, a storage part 144 corresponding to the storage part 44 includes a first storage part 146 storing the avatar and a second storage part 147 storing the Zen space. The avatar and the Zen space are data transmitted from the server 120 via the communication network 18, for example. One of or both the avatar and the Zen space may be previously stored in the terminal apparatus 40 regardless of whether or not to perform Zen, or may also be downloaded from the server 20 to the terminal apparatus 40 every time Zen is performed. In the former case, the data needs not be downloaded every time Zen is performed, and the image generation processing in performing Zen is expected be speeded up. In the latter case, an amount of storage data in the terminal apparatus 40 can be reduced. The question answer data 148 may also be stored in the storage part 144.

The storage part 144 stores a program 145, and a processor 141 executes arithmetic processing in accordance with processing described in the program 145, thereby executing operations as a display control part 142, a chat processing part 151, a display image generation part 152, a question asking part 153, an answer collection part 154, and a time measurement part 155.

The display control part 142 controls display in the display part 47 in the manner similar to the display control part 41a described above. The chat processing part 151 receives input of conversation data from the plurality of members via the plurality of input receiving parts 46 while performing communication with the server 20 in the manner similar to the chat processing part 41b described above, and displays the conversation of the plurality of members in the chat form based on the conversation data.

The display image generation part 152 processes operations as a Zen action image generation part 152a, a Zen action state display image generation part 152b, and a member count image generation part 152c. The Zen action image generation part 152a generates the Zen action image in the manner similar to the Zen action image generation part 22a based on the data regarding the specified Zen space and the specified avatar transmitted from the server 120 via the communication network 18, the data of the avatar, and the data of the Zen space, for example. The generated Zen action image is displayed on the display part 47 in the manner similar to the first embodiment.

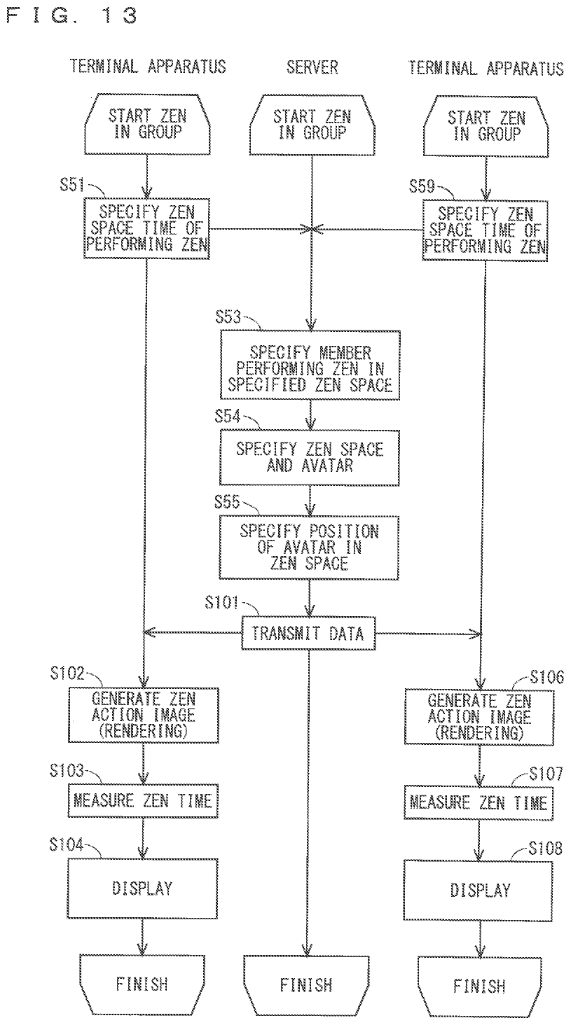

That is to say, the server 120 performs communication with the plurality of terminal apparatuses 40 to execute allocation processing of allocating the avatar in the Zen space, and performs processing relating to the image generation for display in the terminal apparatus 140. For example, as illustrated in FIG. 13, the data determined by the processing in Step S55 in the flow chart illustrated in FIG. 7 may be transmitted to the terminal apparatus 40 corresponding to the member performing Zen in the Zen space in Step S101. The data by the processing in Step S54 may be transmitted to the terminal apparatus 140 to determine the position of the avatar in the Zen space in the terminal apparatus 40. Each terminal apparatus 40 may execute processing of generating the Zen action image (Steps S102 and S106), processing of measuring a time from start of Zen (Steps S103 and S107), and processing of displaying the generated Zen action image on the display part 47 (Steps S104 and S108), for example.

The Zen action state display image generation part 152b executes processing as the Zen action state display image generation part 22b in the first embodiment based on the data acquired in the body state acquisition part 43 of the subject terminal apparatus 140. The member count image generation part 152c executes processing as the member count image generation part 22c in the first embodiment described above based on the data transmitted from the server 120 via the communication network 18.

The question asking part 153, the answer collection part 154, and the time measurement part 155 execute processing as the question asking part 23, the answer collection part 24, and the time measurement part 26 in the first embodiment.

According to the present embodiment, the Zen action image can be generated in the terminal apparatus 140. Thus, processing load of the server 120 can be reduced. After the terminal apparatus 40 receives the data of the Zen space and the avatar, the image data needs not be transmitted and received, thus a data communication amount can be reduced. Even when the data communication is temporality disconnected, the processing of displaying the Zen action image can be continued based on the existing data.

When the terminal apparatus 140 executes the processing as the time measurement part 26, a time of performing Zen can be grasped in the terminal apparatus 140.

### Modification Example

In the first embodiment and the second embodiment described above, some or all of the processing for displaying the Zen action state, the processing for displaying the member count image, and the processing for performing the chat may be omitted. Each processing can be achieved by an optional combination of the servers 20 and 120 and the terminal apparatuses 40 and 140.

Described in each embodiment is the example of the system achieved by the communication between the servers 20 and 120 and the terminal apparatuses 40 and 140, however, a computer in which the system or the program described above may be achieved by performing communication in peer-to-peer form between a plurality of terminal apparatuses.

The server 20 or 120 may have a configuration that some of the parts (for example, the processor 21 and the storage part 30) included in the server 20 or 120 is disposed in a position different from the other parts, and the parts are connected via wired and/or radio communication network.

The processor 21 or 121 may have a configuration that some of the parts (for example, 22 to 27 and 122) included in the processor 21 or 121 is disposed in a position different from the other parts, and the parts are connected via wired and/or radio communication network.

The storage part 30 or 130 may have a configuration that some of the parts (for example, 31 to 35) included in the storage part 30 or 130 is disposed in a position different from the other parts, and the parts are connected via wired and/or radio communication network.

The terminal apparatus 40 or 140 may have a configuration that some of the parts (for example, 41 to 48 and 142 to 155) included in the terminal apparatus 40 or 140 is disposed in a position different from the other parts, and the parts are connected via wired and/or radio communication network. Some of the parts included in the terminal apparatus 40 may be included in the other device such as the server 20.

Each configuration described in each embodiment and modification example can be appropriately combined as long as they are not contradictory.

The present specification and the drawings disclose each aspect described hereinafter.

The first aspect is a Zen facilitation system including: a first storage part storing a plurality of avatars corresponding to a plurality of members belonging to a group performing Zen; a second storage part storing at least one virtual Zen space; at least one input receiving part receiving an input from each of the members; a display image generation part generating a Zen action image in which some of the plurality of avatars perform Zen in the Zen space in accordance with an input regarding a Zen action through the input receiving part; and a display part displaying a Zen action image generated in the display image generation part.

Accordingly, the member sees the plurality of avatars performing Zen in the virtual Zen space through the display part, thereby being able to perform Zen in the actual space while imagining the state of performing Zen in the Zen space. Thus, a mental activity by Zen can be facilitated regardless of a positional, temporal, or epidemiological limitation.

A second aspect is the Zen facilitation system according to the first aspect, wherein the first storage part includes a master avatar as the plurality of avatars, and the display image generation part generates a Zen action image in which some ofthe plurality of avatars including the master avatar perform Zen in the Zen space. Accordingly, the member can perform Zen in the actual space while imagining the state of performing Zen with the master in his/her head.

A third aspect is the Zen facilitation system according to the first or second aspect, including a plurality of terminal apparatuses each including the input receiving part and the display part. Accordingly, the member can perform the input regarding the Zen action using the terminal apparatus, and can further see his/her avatar performing Zen in the virtual Zen space through the terminal apparatus.

A fourth aspect is the Zen facilitation system according to the third aspect, including a server connected to the plurality of terminal apparatuses via communication network and including at least one of the first storage part, the second storage part, and a processing part executing at least some pieces of processing in the display image generation part. Accordingly, the server can perform communication with the plurality of terminal apparatuses to execute the adjustment processing of adjusting the plurality of members.

A fifth aspect is the Zen facilitation system according to any one of the first to fourth aspects, further including: a question asking part providing a question through the display part after the Zen action image is displayed; and an answer collection part collecting an answer inputted through the input receiving part. Accordingly, the question can be provided after the member performs Zen, and the answer can be collected.

A sixth aspect is the Zen facilitation system according to any one of the first to fifth aspects, further including: the plurality of input receiving parts; and a chat processing part receiving input of conversation data from the plurality of members through the plurality of input receiving parts and displaying a conversation of the plurality of members in a chat form based on the conversation data. Accordingly, communication between the members can be facilitated.

A seventh aspect is the Zen facilitation system according to any one of the first to sixth aspects, further including a body state acquisition part acquiring a body state of the member, wherein the display image generation part generates a Zen action state display image based on data acquired by the body state acquisition part. Accordingly, the body state at the time of performing Zen can be easily grasped.

An eighth aspect is a Zen facilitation system according to any one of the first to seventh aspects, further including a time measurement part measuring a time at which the avatar performs Zen in the Zen space in accordance with the input regarding a Zen action through the input receiving part. Accordingly, the time of performing Zen can be grasped.

A ninth aspect is the Zen facilitation system according to any one of the first to eighth aspects, further including an activity member aggregation part counting a total number of members performing Zen at the same time in accordance with the input regarding a Zen action through the input receiving part, wherein the display image generation part generates an image corresponding to the counted number counted by the activity member aggregation part. Accordingly, the number of members performing Zen at the same time is grasped.

A tenth aspect is the Zen facilitation system according to any one of the first to ninth aspects, wherein the second storage part stores the plurality of Zen spaces, and the display image generation part generates a Zen action image in which the avatar performs Zen in one of the plurality of Zen spaces in accordance with the input regarding aZen action through the input receiving part. Accordingly, the member can see the image of his/her avatar performing Zen in one Zen space selected from the plurality of Zen spaces.

An eleventh aspect is the Zen facilitation system according to any one of the first to tenth aspects, wherein the display image generation part generates a Zen action image in which the avatar performs at least one of an action of starting Zen and/or an action of finishing Zen in the Zen space in accordance with the input regarding a Zen action through the input receiving part. In this case, the member sees the action of his/her avatar starting or finishing Zen in the Zen space, thereby being able to easily switch his/her mind before and after performing Zen.

A twelfth aspect is the Zen facilitation system according to any one of the first to eleventh aspects, wherein the Zen action image includes an image of the avatar performing Zen in a still state in the Zen space. Accordingly, the user can easily imagine the state of performing Zen in the still state in the Zen space.

A thirteenth aspect is a program or a computer program medium for making a computer execute: processing of receiving an input regarding a Zen action from a member belonging to a group performing Zen; and processing of displaying, on a display part, a Zen action image in which some of a plurality of avatars corresponding to a plurality of members belonging to the group perform Zen in a virtual Zen space upon receipt of an input regarding a Zen action. Accordingly, the member sees the plurality of avatars performing Zen in the virtual Zen space through the display part, thereby being able to perform Zen in the actual space while imagining the state of performing Zen in the Zen space. Thus, a mental activity by Zen can be facilitated regardless of a positional, temporal, or epidemiological limitation.

A fourteenth aspect is the program or the computer program product according to the thirteenth aspect, wherein the processing of displaying the Zen action image on the display part includes processing of generating a Zen action image in which some of a plurality of avatars corresponding to a plurality of members belonging to a group perform Zen in a virtual Zen space based on a plurality of avatars corresponding to the plurality of members belonging to the group performing Zen and a virtual Zen space. Accordingly, the Zen action image can be generated by the computer displaying the Zen action image.

A fifteenth aspect is the program or the computer program product according to the thirteenth or fourteenth aspects, further making a computer execute: processing of acquiring a body state of the member through a body state acquisition part; and processing of displaying a Zen action state image based on data acquired by the body state acquisition part on the display part. Accordingly, the user can easily grasp the body state at the time of performing Zen.

A sixteenth aspect is the program or the computer program product according to any one of the thirteenth to fifteenth aspects, further making a computer execute processing of measuring a time of performing Zen at which the avatar performs Zen in the Zen space in accordance with an input regarding a Zen action. Accordingly, the user can grasp the time of performing Zen.

A seventeenth aspect is the program or the computer program product according to any one of the thirteenth to sixteenth aspects, further making a computer execute processing of displaying an image corresponding to a total number of members performing Zen at the same time on the display part. Accordingly, the user can grasp the number of members performing Zen at the same time.

An eighteenth aspect is a program or a computer program product for making a computer execute: processing of making a connection communicably with a plurality of terminal apparatuses on a side of a plurality of members belonging to a group performing Zen via communication network; processing of receiving an input regarding a Zen action from each of the members belonging to the group via the plurality of terminal apparatuses; processing of generating data expressing a Zen action image in which some of a plurality of avatars corresponding to the plurality of members belonging to the group perform Zen in a virtual Zen space upon receipt of an input regarding a Zen action; and processing of transmitting the data expressing the Zen action image to at least one of the plurality of terminal apparatuses.

Accordingly, the member sees his/her avatar performing Zen in the virtual Zen space through the terminal apparatus, thereby being able to perform Zen in the actual space while imagining the state of performing Zen in the Zen space in the head. Thus, a mental activity by Zen can be facilitated regardless of a positional, temporal, or epidemiological limitation. The computer can generate data expressing the Zen action image in which at least one of the plurality of avatars performs Zen in the virtual Zen space upon receipt of the input regarding the Zen action from the plurality of terminal apparatuses.

A nineteenth aspect is the program or the computer program product according to the eighteenth aspect: processing of transmitting data expressing the Zen action image to some of the plurality of terminal apparatuses. Accordingly, the plurality of terminal apparatuses share the same image. Thus, the state where the plurality of members perform Zen in the same Zen meditation hall can be displayed at the same time.

A twentieth aspect is the program or the computer program product according to the nineteenth aspect: processing of receiving processing of designating one of a plurality of Zen spaces from each of the members belonging to the group; and processing of generating data expressing a Zen action image in which the avatar performs Zen in one of the plurality of Zen spaces in accordance with an input regarding a Zen action. Accordingly, the member can see the image of his/her avatar performing Zen in one Zen space selected from the plurality of Zen spaces. In this case, when the other member designates the same Zen space, the avatar corresponding to the other member and performing Zen in the same Zen space can also be observed.

The foregoing description is in all aspects illustrative, thus the present invention is not limited thereto. It is therefore understood that numerous modification examples can be devised without departing from the scope of the invention.

### EXPLANATION OF REFERENCE SIGNS

10 Zen facilitation system
18 communication network
20, 120 server
21, 121 processor
22, 152 display image generation part
22a, 152a Zen action image generation part
22b. 152b Zen action state display image generation part
22c, 152c member count image generation part
23, 153 question asking part
24, 154 answer collection part
25 chat relay processing part
26, 155 time measurement part
27 activity member aggregation part
30 storage part
31, 146 first storage part
31a, 31b avatar
31c master avatar
32, 147 second storage part
32a Zen space
35, 135 program
40, 40A, 40B, 40C, 40D, 40E, 40F, 40G, 140 terminal apparatus
41, 141 processor
41a, 142 display control part
41b, 151 chat processing part
43 body state acquisition part
44, 144 storage part
45 program
46 input receiving part
47 display part
122 Zen space avatar specifying part

1. A Zen facilitation system, comprising:
a first storage storing a plurality of avatars corresponding to a plurality of members belonging to a group performing Zen;
a second storage storing at least one virtual Zen space;
at least one input receiving part receiving an input from each of the members;
a processor generating a Zen action image in which some of the plurality of avatars perform Zen in the Zen space in accordance with an input regarding a Zen action through the input receiving part; and
a display displaying a Zen action image generated in processor.

2. The Zen facilitation system according to claim 1, wherein
the first storage stores a master avatar as at least of the plurality of avatars, and
the processor generates a Zen action image in which some of the plurality of avatars including the master avatar perform Zen in the Zen space.

3. The Zen facilitation system according to claim 1, comprising
a plurality of terminal apparatuses including the input receiving part and the display.

4. The Zen facilitation system according to claim 3, comprising
a server connected to the plurality of terminal apparatuses via communication network and including at least one of the first storage, the second storage, or a processing part executing at least some pieces of processing in the processor.

5. The Zen facilitation system according to claim 1, further comprising
a question asking part providing a question through the display after the Zen action image is displayed; and
an answer collection part collecting an answer inputted through the input receiving part.

6. The Zen facilitation system according to claim 1, further comprising:
the plurality of input receiving parts; and
a chat processing part receiving input of conversation data from the plurality of members through the plurality of input receiving parts and displaying a conversation of the plurality of members in a chat form based on the conversation data.

7. The Zen facilitation system according to claim 1, further comprising
a body state acquisition part acquiring a body state of the member, wherein
the processor generates a Zen action state display image based on data acquired by the body state acquisition part.

8. The Zen facilitation system according to claim 1, further comprising
a time measurement part measuring a time at which the avatar performs Zen in the Zen space in accordance with an input regarding a Zen action through the input receiving part.

9. The Zen facilitation system according to claim 1, further comprising
an activity member aggregation part counting a total number of members performing Zen at a same time in accordance with an input regarding a Zen action through the input receiving part, wherein
the processor generates an image corresponding to the counted number counted by the activity member aggregation part.

10. The Zen facilitation system according to claim 1, wherein
the second storage stores the plurality of Zen spaces, and
the processor generates a Zen action image in which the avatar performs Zen in one of the plurality of Zen spaces in accordance with an input regarding a Zen action through the input receiving part.

11. The Zen facilitation system according to claim 1, wherein
the processor generates a Zen action image in which the avatar performs at least one of an action of starting Zen and/or an action of finishing Zen in the Zen space in accordance with an input regarding a Zen action through the input receiving part.

12. The Zen facilitation system according to claim 1, wherein
the Zen action image includes an image of the avatar performing Zen in a still state in the Zen space.

13. A non-transitory computer-readable storage medium storing a program for making a computer executes:
processing of receiving an input regarding a Zen action from a member belonging to a group performing Zen; and
processing of displaying, on a display, a Zen action image in which some of a plurality of avatars corresponding to a plurality of members belonging to the group perform Zen in a virtual Zen space upon receipt of an input regarding a Zen action.

14. The non-transitory computer-readable storage medium according to claim 13, wherein
the processing of displaying the Zen action image on the display includes processing of generating a Zen action image in which some of a plurality of avatars corresponding to a plurality of members belonging to a group perform Zen in a virtual Zen space based on a plurality of avatars corresponding to the plurality of members belonging to the group performing Zen and a virtual Zen space.

15. The non-transitory computer-readable storage medium according to claim 13, the program further making a computer execute:
processing of acquiring a body state of the member through a body state acquisition part; and
processing of displaying a Zen action state image based on data acquired by the body state acquisition part on the display.

16. The non-transitory computer-readable storage medium according to claim 13, the program further making a computer execute;
processing of measuring a time of performing Zen at which the avatar performs Zen in the Zen space in accordance with an input regarding a Zen action.

17. The non-transitory computer-readable storage medium according to claim 13, the program further making a computer execute;
processing of displaying an image corresponding to a total number of members performing Zen at a same time on the display.

18. A non-transitory computer-readable storage medium storing a program for making a computer execute:
processing of making a connection communicably with a plurality of terminal apparatuses on a side of a plurality of members belonging to a group performing Zen via communication network;
processing of receiving an input regarding a Zen action from each of the members belonging to the group via the plurality of terminal apparatuses;
processing of generating data expressing a Zen action image in which some of a plurality of avatars corresponding to the plurality of members belonging to the group perform Zen in a virtual Zen space upon receipt of an input regarding a Zen action; and
processing of transmitting the data expressing the Zen action image to at least one of the plurality of terminal apparatuses.

19. The non-transitory computer-readable storage medium according to claim 18, the program further making a computer execute:
processing of transmitting data expressing the Zen action image to some of the plurality of terminal apparatuses.

20. The non-transitory computer-readable storage medium according to claim 19, the program further making a computer execute:
processing of receiving processing of designating one of a plurality of Zen spaces from each of the members belonging to the group; and
processing of generating data expressing a Zen action image in which the avatar performs Zen in one of the plurality of Zen spaces in accordance with an input regarding a Zen action.
